(19) United States
(12) Patent Application Publication
(10) Pub. No.: US 2026/0223911 A1
(43) Pub. Date: Aug. 6, 2026

(54) ATOMIZING LIQUID MODIFIER, ATOMIZING LIQUID AND AEROSOL GENERATING DEVICE

(71) Applicant: SHENZHEN YUPENG TECHNOLOGY CO., LTD., Shenzhen (CN)

(72) Inventors: Weibo LI, Shenzhen (CN); Qiaoxia ZHONG, Shenzhen (CN); Hanmou YU, Shenzhen (CN); Junfei XIAO, Shenzhen (CN); Shaohua LIU, Shenzhen (CN); Zhonghu ZHANG, Shenzhen (CN)

(73) Assignee: SHENZHEN YUPENG TECHNOLOGY CO., LTD., Shenzhen (CN)

(21) Appl. No.: 19/564,112

(22) Filed: Mar. 11, 2026

Related U.S. Application Data
(63) Continuation of application No. PCT/CN2023/132330, filed on Nov. 17, 2023.

(30) Foreign Application Priority Data
Sep. 27, 2023 (CN) 202311263198.8

(51) Int. Cl. A24B 15/32; A61K 47/10; A61K 47/22; A61K 47/26; A61K 47/28

(52) U.S. Cl. CPC A24B 15/32; A61K 47/10; A61K 47/22; A61K 47/26; A61K 47/28

(57) ABSTRACT

The present application discloses an atomizing liquid modifier, an atomizing liquid, and an aerosol generating device. The atomizing liquid modifier includes 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl)chroman-4-one, 3-hydroxy-citronellolic acid 3-O-β-D-glucopyranoside, 3′,4′,5,6,7,8-hexamethoxyflavone, and antioxidants.

### CROSS-REFERENCE TO RELATED APPLICATIONS

The present application is a continuation application of International Application No. PCT/CN2023/132330, filed on Nov. 17, 2023, which claims priority to Chinese Patent Application No. 202311263198.8, filed on Sep. 27, 2023. The disclosures of the above-mentioned applications are incorporated herein by reference in their entireties.

### TECHNICAL FIELD

The present application relates to the technical field of fragrances, and in particular to an atomizing liquid modifier, an atomizing liquid, and an aerosol generating device.

### BACKGROUND

Atomizing liquid is a liquid used in aerosol generating devices to produce vapor. It typically consists of propylene glycol (PG), glycerol (VG), flavorings, nicotine, and other additives. Normally, saliva secretion is regulated by nerves and hormones, contributing to oral moisture, digestion, and protection. Certain chemicals can interfere with saliva secretion in different ways, leading to reduced saliva production. Propylene glycol and glycerol, commonly found in the atomizing liquid, are two such examples. As common base ingredients, long-term use of propylene glycol and glycerol can disrupt saliva secretion, causing chronic dry mouth. This not only affects user comfort and experience but may also negatively impact oral health. Therefore, addressing the dry mouth caused by the use of atomizing liquids can optimize the user experience.

### SUMMARY

The main purpose of the present application is to propose an atomizing liquid modifier that aims to overcome the long-term oral dryness problem caused by the use of atomizing liquids in the related art.

To achieve the above purpose, the present application proposes an atomizing liquid modifier, including the following raw material components:

6-O-p-hydroxybenzoyl catalpol, 7α-hydroxy-β-sitosterol, 5,7-dihydroxy-6,8-dimethyl-3-(4′-hydroxy-3′,5′-dimethoxybenzyl)chroman-4-one, 3-hydroxy-citronellolic acid 3-O-β-D-glucopyranoside, 3′,4′,5,6,7,8-hexamethoxyflavone, and an antioxidant.

In an embodiment, the antioxidant includes resveratrol.

In an embodiment, by mass parts, the atomizing liquid modifier includes the following components:
0.10 to 1 parts of 6-O-p-hydroxybenzoyl catalpol;
0.1 to 1 parts of 7α-hydroxy-β-sitosterol;
0.1 to 0.5 parts of 5,7-dihydroxy-6,8-dimethyl-3-(4′-hydroxy-3′,5′-dimethoxybenzyl)chroman-4-one;
0.1 to 0.5 parts of 3-hydroxycitronellolic acid 3-O-β-D-glucopyranoside;
0.01 to 0.05 parts of resveratrol; and
0.01 to 0.05 parts of 3′,4′,5,6,7,8-hexamethoxyflavone and antioxidants.

The present application also proposes a use of an atomizing liquid modifier in the long-term relief of oral dryness caused by the use of atomizing liquid. The atomizing liquid modifier includes the following raw material components: 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl) chroman-4-one, 3-hydroxy-citronellolic acid 3-O-β-D-glucopyranoside, 3′,4′,5,6,7,8-hexamethoxyflavone, and an antioxidant.

The present application also proposes an atomizing liquid, including the aforementioned atomizing liquid modifier.

In an embodiment, the atomizing liquid further includes glycerol and 1,2-propanediol.

In an embodiment, the mass ratio of glycerol to atomizing liquid modifier is 10:(0.084 to 0.62).

The present application also proposes an aerosol generating device, including the aforementioned atomizing liquid.

The atomizing liquid modifiers provided in the present application include 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl)chroman-4-one, 3-hydroxy-citronellolic acid 3-O-β-D-glucopyranoside, 3′,4′,5,6,7,8-hexamethoxyflavone, and antioxidants. 6-O-p-hydroxybenzoyl catalpol is naturally derived from various plants in the Bignoniaceae and Plantaginaceae families, such as Oroxylum indicum and Plantago asiatica. NF-κB is a key nuclear transcription factor involved in various cellular activities, including immune cell activation, T and B lymphocyte development, stress response, and apoptosis. It can regulate many immune and inflammation-related genes and plays a role in cell damage repair. Catalpol is an iridoid glucoside that regulates NF-κB. Changes in its functional group often alter the properties of the compound. Compounds with an acyl substitution at the 6-O position, namely 6-O-acyl catalpol, exhibit stronger NF-κB regulatory activity compared to catalpol. 7α-hydroxy-β-sitosterol, naturally derived from various Ranunculaceae plants, promotes cell regeneration. 5,7-Dihydroxy-6,8-dimethyl-3-(4′-hydroxy-3′,5′-dimethoxybenzyl)chroman-4-one possesses potent free radical scavenging properties. 3-hydroxy-citronellolic acid 3-O-β-D-glucopyranoside may interfere with multiple inflammatory signaling pathways, such as the NF-κB pathway and mitochondrial-mediated pathways, and can inhibit inflammatory responses by suppressing NF-κB activation. 3′,4′,5,6,7,8-hexamethoxyflavone, naturally derived from citrus fruits such as lime and grapefruit, possess antioxidant activity. By combining the above-mentioned substances, oral inflammation or cell damage caused by propylene glycol and glycerol can be reduced, promoting the recovery of affected cells or the elimination of inflammation, thereby alleviating the reduction in saliva secretion caused by propylene glycol and glycerol. The atomizing liquid modifier provided in the present application has a long-lasting effect in relieving oral dryness caused by atomizing liquids.

### DETAILED DESCRIPTION OF THE EMBODIMENTS

To make the objectives, technical solutions, and advantages of the embodiments of the present application clearer, the technical solutions in the embodiments of the present application will be clearly and completely described below. Where specific conditions are not specified in the embodiments, conventional conditions or conditions recommended by the manufacturer shall apply. Where the manufacturers of reagents or instruments are not specified, they are all conventional products that can be purchased commercially. Furthermore, the meaning of “and/or” throughout the text includes three parallel solutions; for example, “A and/or B” includes solution A, solution B, or a solution where both A and B are satisfied. In addition, the technical solutions of the various embodiments can be combined with each other, but this must be based on the ability of those skilled in the art to implement them. When the combination of technical solutions is contradictory or cannot be implemented, it should be considered that such a combination of technical solutions does not exist and is not within the scope of the present application. Based on the embodiments in the present application, all other embodiments obtained by those skilled in the art without creative effort are within the scope of the present application.

Atomizing liquid is a liquid used in aerosol generating devices to produce vapor. It is typically composed of propylene glycol (PG), glycerol (VG), flavorings, nicotine, and other additives. Normally, saliva secretion is regulated by nerves and hormones, contributing to oral moisture, digestion, and protection. Certain chemicals can interfere with saliva secretion in different ways, leading to reduced saliva production. Propylene glycol and glycerol, commonly found in the atomizing liquid, are two such examples. As common base ingredients, long-term use of propylene glycol and glycerol can disrupt saliva secretion, causing chronic dry mouth. This not only affects user comfort and experience but may also negatively impact oral health. Therefore, addressing the dry mouth caused by atomizing liquids can optimize the user experience.

In view of this, the present application proposes an atomizing liquid modifier to overcome the problem of dry mouth caused by the atomizing liquid in the related art. The raw material components of the atomizing liquid modifier include 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl)chroman-4-one, 3-hydroxy-citronellolic acid 3-O-β-D-glucopyranoside, 3′,4′,5,6,7,8-hexamethoxyflavone, and antioxidants.

The atomizing liquid modifier provided in the present application includes 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl)chroman-4-one, 3-hydroxy-citronellolic acid 3-O-β-D-glucopyranoside, 3′,4′,5,6,7,8-hexamethoxyflavone, and antioxidants. 6-O-p-hydroxybenzoyl catalpol is naturally derived from various plants in the Bignoniaceae and Plantaginaceae families, such as *Oroxylum indicum* and *Plantago asiatica*. NF-κB is a key nuclear transcription factor involved in various cellular activities, including immune cell activation, T and B lymphocyte development, stress response, and apoptosis. It can regulate many immune and inflammation-related genes and plays a role in cell damage repair. Catalpol is an iridoid glucoside that regulates NF-κB. Changes in functional groups often alter the properties of the compound; compounds with an acyl substitution at the 6-O position, namely 6-O-acyl catalpol, exhibit stronger NF-κB regulatory activity. 7α-hydroxy-β-sitosterol, naturally derived from various Ranunculaceae plants, promotes cell regeneration. 5,7-Dihydroxy-6,8-dimethyl-3-(4′-hydroxy-3′,5′-dimethoxybenzyl)chroman-4-one possesses potent free radical scavenging properties. 3-hydroxy-citronellolic acid 3-O-β-D-glucopyranoside may interfere with multiple inflammatory signaling pathways, such as the NF-κB pathway and mitochondrial-mediated pathways, and can inhibit inflammatory responses by suppressing NF-κB activation. 3′,4′,5,6,7,8-hexamethoxyflavone, naturally derived from citrus fruits such as lime and grapefruit, possess antioxidant activity. By combining the above-mentioned substances, oral inflammation or cell damage caused by propylene glycol and glycerol can be reduced, promoting the recovery of affected cells or the elimination of inflammation, thereby alleviating the reduction in saliva secretion caused by propylene glycol and glycerol. The atomizing liquid modifier provided in the present application has a long-lasting effect in relieving oral dryness caused by atomizing liquid.

The antioxidants include resveratrol. Resveratrol is naturally derived from grape skins and is an important physiologically active polyphenol, a major active ingredient in many plants. Other antioxidants such as quercetin can also be used, but their combined effect with other ingredients in alleviating dry mouth caused by atomizing liquid is not as good as that of resveratrol. Therefore, in an embodiment of the present application, resveratrol is used to further and more effectively alleviate dry mouth caused by atomizing liquid.

Further, by weight, the atomizing liquid modifier includes the following components: 0.10 to 1 parts of 6-O-p-hydroxybenzoyl catalpol; 0.1 to 1 parts of 7α-hydroxy-β-sitosterol; 0.1 to 0.5 parts of 5,7-dihydroxy-6,8-dimethyl-3-(4′-hydroxy-3′,5′-dimethoxybenzyl)chroman-4-one; 0.1 to 0.5 parts of 3-hydroxy-citronellolic acid 3-O-β-D-glucopyranoside; 0.01 to 0.05 parts of resveratrol; 0.01 to 0.05 parts of 3′,4′,5,6,7,8-hexamethoxyflavone; and 0.01 to 0.05 parts of antioxidant. Using the above weight fractions can better and more effectively alleviate the problem of dry mouth caused by the atomizing liquid.

The present application also proposes a use of an atomizing liquid modifier in the long-term relief of dry mouth caused by the atomizing liquid. The atomizing liquid modifier includes the following raw material components: 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl) chroman-4-one, 3-hydroxy-citronellolic acid 3-O-β-D-glucopyranoside, 3′,4′,5,6,7,8-hexamethoxyflavone, and an antioxidant. The use of the atomizing liquid modifier proposed in the present application in the long-term relief of dry mouth caused by the atomizing liquid can achieve a long-term relief of the dry mouth problem caused by the atomizing liquid.

The present application also proposes an atomizing liquid, including the aforementioned atomizing liquid modifier. The atomizing liquid possesses all the technical solutions of the aforementioned atomizing liquid modifier, and therefore has all the beneficial effects of the aforementioned atomizing liquid modifier, which will not be repeated here.

Furthermore, the atomizing liquid also includes glycerol and 1,2-propanediol, and a mass ratio of glycerol to atomizing liquid modifier is 10:(0.084~0.62). At this mass ratio, the atomizing liquid modifier provides better long-lasting relief for dry mouth caused by the atomizing liquid.

The method for preparing the atomizing liquid proposed in the present application is as follows: the above-mentioned atomizing liquid modifier is added to glycerol, then 1,2-propanediol is added to obtain a mixture, and the mixture is stirred for 5 minutes to obtain the atomizing liquid.

The present application also proposes an aerosol generating device, including the aforementioned atomizing liquid. The aerosol generating device possesses all the technical solutions of the aforementioned atomizing liquid modifier, and therefore has all the beneficial effects of the aforementioned atomizing liquid modifier, which will not be repeated here.

The technical solution of the present application will be further described in detail below with reference to specific embodiments. It should be understood that the following embodiments are only used to explain the present application and are not intended to limit the present application.

### Example 1

The raw material components of the atomizing liquid modifier are: 6-O-p-hydroxybenzoyl catalpol, 7α-hydroxy-β-sitosterol, 5,7-dihydroxy-6,8-dimethyl-3-(4′-hydroxy-3′,5′-dimethoxybenzyl)chroman-4-one, 3-hydroxy-citronellolic acid 3-O-β-D-glucopyranoside, 3′,4′,5,6,7,8-hexamethoxyflavone, and resveratrol.

### Example 2

The raw material components and corresponding mass fractions of the atomizing liquid modifier are as follows: 0.05 parts of 6-O-p-hydroxybenzoyl catalpol, 0.05 parts of 7α-hydroxy-β-sitosterol, 0.01 parts of 5,7-dihydroxy-6,8-dimethyl-3-(4′-hydroxy-3′,5′-dimethoxybenzyl)chroman-4-one, 0.01 parts of 3-hydroxy-citronellolic acid 3-O-β-D-glucopyranoside, 0.001 parts of 3′,4′,5,6,7,8-hexamethoxyflavone, and 0.001 parts of resveratrol.

### Example 3

The raw material components and corresponding mass fractions of the atomizing liquid modifier are as follows: 0.1 parts of 6-O-p-hydroxybenzoyl catalpol, 0.1 parts of 7α-hydroxy-β-sitosterol, 0.05 parts of 5,7-dihydroxy-6,8-dimethyl-3-(4′-hydroxy-3′,5′-dimethoxybenzyl)chroman-4-one, 0.05 parts of 3-hydroxy-citronellolic acid 3-O-β-D-glucopyranoside, 0.005 parts of 3′,4′,5,6,7,8-hexamethoxyflavone, and 0.005 parts of resveratrol.

### Example 4

The raw material components and corresponding mass fractions of the atomizing liquid modifier are as follows: 0.2 parts of 6-O-p-hydroxybenzoyl catalpol, 0.2 parts of 7α-hydroxy-β-sitosterol, 0.1 parts of 5,7-dihydroxy-6,8-dimethyl-3-(4′-hydroxy-3′,5′-dimethoxybenzyl)chroman-4-one, 0.1 parts of 3-hydroxy-citronellolic acid 3-O-β-D-glucopyranoside, 0.01 parts of 3′,4′,5,6,7,8-hexamethoxyflavone, and 0.01 parts of resveratrol.

### Example 5

The raw material components and corresponding mass fractions of the atomizing liquid modifier are as follows: 1 parts of 6-O-p-hydroxybenzoyl catalpol, 1 parts of 7α-hydroxy-β-sitosterol, 0.5 parts of 5,7-dihydroxy-6,8-dimethyl-3-(4′-hydroxy-3′,5′-dimethoxybenzyl)chroman-4-one, 0.5 parts of 3-hydroxy-citronellolic acid 3-O-β-D-glucopyranoside, 0.05 parts of 3′,4′,5,6,7,8-hexamethoxyflavone, and 0.05 parts of resveratrol.

### Example 6

The raw material components and corresponding mass fractions of the atomizing liquid modifier are as follows: 0.1 parts of 6-O-p-hydroxybenzoyl catalpol, 0.1 parts of 7α-hydroxy-β-sitosterol, 0.2 parts of 5,7-dihydroxy-6,8-dimethyl-3-(4′-hydroxy-3′,5′-dimethoxybenzyl)chroman-4-one, 0.2 parts of 3-hydroxy-citronellolic acid 3-O-β-D-glucopyranoside, 0.02 parts of 3′,4′,5,6,7,8-hexamethoxyflavone, and 0.02 parts of resveratrol.

### Example 7

The atomizing liquid consists of: 50 parts glycerol, 0.05 parts 6-O-p-hydroxybenzoyl catalpol, 0.05 parts 7α-hydroxy-β-sitosterol, 0.01 parts 5,7-dihydroxy-6,8-dimethyl-3-(4′-hydroxy-3′,5′-dimethoxybenzyl) chroman-4-one, 0.01 parts 3-hydroxy-citronellolic acid 3-O-β-D-glucopyranoside, 0.001 parts 3′,4′,5,6,7,8-hexamethoxyflavone, 0.001 parts resveratrol, and 49.878 parts 1,2-propanediol.

### Example 8

The atomizing liquid consists of: 50 parts glycerol, 0.1 parts 6-O-p-hydroxybenzoyl catalpol, 0.1 parts 7α-hydroxy-β-sitosterol, 0.05 parts 5,7-dihydroxy-6,8-dimethyl-3-(4′-hydroxy-3′,5′-dimethoxybenzyl) chroman-4-one, 0.05 parts 3-hydroxy-citronellolic acid 3-O-β-D-glucopyranoside, 0.005 parts 3′,4′,5,6,7,8-hexamethoxyflavone, 0.005 parts resveratrol, and 49.69 parts 1,2-propanediol.

### Example 9

The atomizing liquid consists of: 50 parts glycerol, 0.2 parts 6-O-p-hydroxybenzoyl catalpol, 0.2 parts 7α-hydroxy-β-sitosterol, 0.1 parts 5,7-dihydroxy-6,8-dimethyl-3-(4′-hydroxy-3′,5′-dimethoxybenzyl) chroman-4-one, 0.1 parts 3-hydroxy-citronellolic acid 3-O-β-D-glucopyranoside, 0.01 parts 3′,4′,5,6,7,8-hexamethoxyflavone, 0.01 parts resveratrol, and 49.38 parts 1,2-propanediol.

### Example 10

The atomizing liquid consists of: 50 parts glycerol, 1 parts of 6-O-p-hydroxybenzoyl catalpol, 1 parts of 7α-hydroxy-β-sitosterol, 0.5 parts of 5,7-dihydroxy-6,8-dimethyl-3-(4′-hydroxy-3′,5′-dimethoxybenzyl) chroman-4-one, 0.5 parts of 3-hydroxy-citronellolic acid 3-O-3-D-glucopyranoside, 0.05 parts of 3′,4′,5,6,7,8-hexamethoxyflavone, 0.05 parts resveratrol, and 46.9 parts of 1,2-propanediol.

### Example 11

The atomizing liquid consists of: 50 parts of glycerol, 0.1 parts of 6-O-p-hydroxybenzoyl catalpol, 0.1 parts of 7α-hydroxy-β-sitosterol, 0.2 parts of 5,7-dihydroxy-6,8-dimethyl-3-(4′-hydroxy-3′,5′-dimethoxybenzyl) chroman-4-one, 0.2 parts of 3-hydroxy-citronellolic acid 3-O-β-D-glucopyranoside, 0.02 parts of 3′,4′,5,6,7,8-hexamethoxyflavone, 0.02 parts of resveratrol, and 49.36 parts of 1,2-propanediol.

### Comparative Example 1

The atomizing liquid consists of 50 parts of glycerol and 50 parts of 1,2-propanediol.

### Comparative Example 2

The atomizing liquid consists of: 50 parts of glycerol, 0.1 parts of 7α-hydroxy-β-sitosterol, 0.05 parts of 5,7-dihydroxy-6,8-dimethyl-3-(4′-hydroxy-3′,5′-dimethoxybenzyl)chroman-4-one, 0.05 parts of 3-hydroxy-citronellolic acid 3-O-β-D-glucopyranoside, 0.005 parts of 3′,4′,5,6,7,8-hexamethoxyflavone, 0.005 parts of resveratrol, and 49.79 parts of 1,2-propanediol.

### Comparative Example 3

The atomizing liquid consists of: 50 parts of glycerol, 0.1 parts of 6-O-p-hydroxybenzoyl catalpol, 0.05 parts of 5,7-dihydroxy-6,8-dimethyl-3-(4′-hydroxy-3′,5′-dimethoxybenzyl) chroman-4-one, 0.05 parts of 3-hydroxy-citronellolic acid 3-O-β-D-glucopyranoside, 0.005 parts of 3′,4′,5,6,7,8-hexamethoxyflavone, 0.005 parts of resveratrol, and 49.79 parts of 1,2-propanediol.

### Comparative Example 4

The atomizing liquid consists of: 50 parts of glycerol, 0.1 parts of 6-O-p-hydroxybenzoyl catalpol, 0.1 parts of 7α-hydroxy-β-sitosterol, 0.05 parts of 3-hydroxy-citronellolic acid 3-O-β-D-glucopyranoside, 0.005 parts of 3′,4′,5,6,7,8-hexamethoxyflavone, 0.005 parts of resveratrol, and 49.74 parts of 1,2-propanediol.

### Comparative Example 5

The atomizing liquid consists of: 50 parts of glycerol, 0.1 parts of 6-O-p-hydroxybenzoyl catalpol, 0.1 parts of 7α-hydroxy-β-sitosterol, 0.05 parts of 5,7-dihydroxy-6,8-dimethyl-3-(4′-hydroxy-3′,5′-dimethoxybenzyl)chroman-4-one, 0.005 parts of 3′,4′,5,6,7,8-hexamethoxyflavone, 0.005 parts of resveratrol, and 49.74 parts of 1,2-propanediol.

### Comparative Example 6

The atomizing liquid consists of: 50 parts of glycerol, 0.1 parts of 6-O-p-hydroxybenzoyl catalpol, 0.1 parts of 7α-hydroxy-β-sitosterol, 0.05 parts of 5,7-dihydroxy-6,8-dimethyl-3-(4′-hydroxy-3′,5′-dimethoxybenzyl) chroman-4-one, 0.05 parts of 3-hydroxy-citronellolic acid 3-O-β-D-glucopyranoside, 0.005 parts of resveratrol, and 49.695 parts of 1,2-propanediol.

### Comparative Example 7

The atomizing liquid consists of: 50 parts of glycerol, 0.1 parts of 6-O-p-hydroxybenzoyl catalpol, 0.1 parts of 7α-hydroxy-β-sitosterol, 0.05 parts of 5,7-dihydroxy-6,8-dimethyl-3-(4′-hydroxy-3′,5′-dimethoxybenzyl) chroman-4-one, 0.05 parts of 3-hydroxy-citronellolic acid 3-O-β-D-glucopyranoside, 0.005 parts of 3′,4′,5,6,7,8-hexamethoxyflavone, and 49.695 parts of 1,2-propanediol.

### Test Methods and Results

### 1. Preparations Before Testing:

Healthy male SD rats aged 7-8 weeks and weighing 180-200 g were randomly divided into 13 groups, with 6 rats in each group.

The atomizing liquids obtained in Examples 7-11 and Comparative Examples 1-7 were added to the aerosol generating device, and aerosols were released by heating after being powered on. 35 ml of aerosol was released each time, and 0.3 ml was diverted to the oral aerosol delivery system and delivered to the oral cavity of rats in groups 1-12. Treatment was performed every 30 seconds, 200 times per day, for 7 days.

The rats in group 13 were not treated with the atomizing liquid and served as the blank control group.

### 2. Detection Indicators and Methods:

Indicators: the detection indicators were the salivation volume of rats in each group 1 day before treatment and 3, 7, 15, and 30 days after treatment. The indicator for 1 day before treatment served as a control group within each group.

Methods: Before measurement, sterile defatted cotton balls were prepared and their dry weight was measured to be 26±0.17 mg. When measuring saliva secretion, the pre-weighed cotton balls were placed in the rat's cheek, and the amount of saliva secreted by the rat was measured. Each cotton ball was replaced every 30 minutes, and the measurement was continued for 60 minutes. The maximum saliva secretion was calculated by the weight difference of the dry cotton balls before and after weighing.

Saliva secretion (mg)=Wet weight of dry cotton ball−Dry weight of dry cotton ball

The test results are shown in Table 1:

TABLE 1
Saliva secretion of rats in Examples 7-11, Comparative Examples 1-7, and the
control group for 3-30 days

| | Saliva | Saliva | Saliva | Saliva | Saliva |
|---|---|---|---|---|---|
| | production (mg) | production on | production on | production on | production on |
| Group | 1 day ago | day 3 (mg) | day 7 (mg) | day 15 (mg) | day 30 (mg) |
| Example 7 | 46.832 ± 0.635 | 40.790 ± 2.035 | 41.059 ± 0.779 | 43.006 ± 1.302 | 45.65 ± 0.705 |
| Example 8 | 46.755 ± 0.833 | 48.713 ± 1.732 | 46.531 ± 2.232 | 46.706 ± 2.170 | 46.355 ± 1.325 |
| Example 9 | 46.527 ± 0.751 | 51.235 ± 1.325 | 48.772 ± 1.059 | 47.655 ± 2.033 | 46.324 ± 1.652 |
| Example 10 | 46.468 ± 0.562 | 52.941 ± 0.524 | 49.168 ± 1.269 | 47.882 ± 1.924 | 46.559 ± 1.376 |
| Example 11 | 46.665 ± 0.453 | 50.238 ± 1.056 | 48.367 ± 0.876 | 46.934 ± 1.575 | 46.468 ± 1.253 |
| Comparative | 46.650 ± 0.770 | 33.677 ± 1.337 | 33.860 ± 0.673 | 34.179 ± 0.892 | 35.166 ± 1.246 |
| Example 1 | | | | | |
| Comparative | 46.395 ± 0.739 | 36.862 ± 1.468 | 37.254 ± 0.964 | 38.345 ± 2.376 | 40.798 ± 1.364 |
| Example 2 | | | | | |
| Comparative | 46.640 ± 0.784 | 35.397 ± 1.285 | 36.834 ± 1.642 | 37.788 ± 1.228 | 39.354 ± 0.879 |
| Example 3 | | | | | |
| Comparative | 46.775 ± 0.529 | 36.840 ± 0.862 | 36.982 ± 0.952 | 38.247 ± 0.736 | 38.657 ± 1.523 |
| Example 4 | | | | | |
| Comparative | 46.628 ± 1.063 | 37.695 ± 0.936 | 37.924 ± 1.364 | 38.445 ± 1.865 | 39.248 ± 0.617 |
| Example 5 | | | | | |
| Comparative | 46.673 ± 0.517 | 36.670 ± 1.253 | 37.456 ± 2.642 | 39.673 ± 0.927 | 40.213 ± 2.165 |
| Example 6 | | | | | |
| Comparative | 46.458 ± 0.842 | 38.177 ± 1.168 | 38.768 ± 0.954 | 40.354 ± 1.160 | 42.786 ± 1.395 |
| Example 7 | | | | | |
| Control Group | 46.779 ± 0.634 | 46.880 ± 0.873 | 46.578 ± 0.993 | 46.856 ± 1.032 | 46.602 ± 1.005 |

TABLE 2
Percentage increase in saliva secretion in rats of Examples
7-11, Comparative Examples 1-7, and the control group after
treatment for 3-30 days compared to before treatment

| | Percentage | Percentage | Percentage | Percentage |
|---|---|---|---|---|
| | increase | increase | increase | increase |
| | in saliva | in saliva | in saliva | in saliva |
| | production | production | production | production |
| Group | on day 3 | on day 7 | on day 15 | on day 30 |
| Example 7 | −12.90% | −12.33% | −8.17% | −2.52% |
| Example 8 | 4.19% | −0.48% | −0.10% | −0.86% |
| Example 9 | 10.12% | 4.83% | 2.42% | −0.44% |
| Example 10 | 13.93% | 5.81% | 3.04% | 0.20% |
| Example 11 | 7.66% | 3.65% | 0.58% | −0.42% |
| Comparative | −27.81% | −27.42% | −26.73% | −24.62% |
| Example 1 | | | | |
| Comparative | −20.55% | −19.70% | −17.35% | −12.06% |
| Example 2 | | | | |
| Comparative | −24.11% | −21.02% | −18.98% | −15.62% |
| Example 3 | | | | |
| Comparative | −21.24% | −20.94% | −18.23% | −17.36% |
| Example 4 | | | | |
| Comparative | −19.16% | −18.67% | −17.55% | −15.83% |
| Example 5 | | | | |
| Comparative | −21.43% | −19.75% | −15.00% | −13.84% |
| Example 6 | | | | |
| Comparative | −17.82% | −16.55% | −13.14% | −7.90% |
| Example 7 | | | | |
| Control Group | 0.22% | −0.43% | 0.16% | −0.38% |

There was no significant difference in saliva secretion among the control group rats that were not treated with the atomizing liquid before day 1 and on days 3, 7, 15 and 30, indicating that under the same living conditions, the saliva secretion of rats that did not receive any treatment remained stable.

In Example 11, the saliva secretion of rats treated with the atomizing liquid showed a significant difference between the amount of saliva secretion one day after treatment and the amount of saliva secretion one day before treatment, in the results over 3-30 days. This indicates that the aerosol in the control group affected the saliva secretion of rats, that is, propylene glycol and glycerol caused saliva secretion disorder in rats, and this effect lasted for a period of time.

The difference in saliva secretion of rats treated with the atomizing liquid prepared in Examples 7-10 was within 5 mg after treatment compared with that 1 day prior to treatment. The saliva secretion of rats treated with the atomizing liquid prepared in Example 7 decreased slightly within 15 days, but compared with rats treated with the atomizing liquid prepared in Comparative Example 1, it played a greater role in alleviating the reduction of saliva secretion.

The rats treated with the atomizing liquid prepared in Examples 8-10 showed increased saliva secretion within 7 days compared to the amount of saliva secreted 1 day before treatment. By day 15, normal saliva secretion function was restored and continued until day 30. Compared with the rats treated with the atomizing liquid prepared in Comparative Example 1, it had a greater and more sustained effect in alleviating the reduction in saliva secretion.

In summary, the atomizing liquid modifier provided in the present application can alleviate saliva secretion disorders caused by propylene glycol and glycerol in the atomizing liquid and can provide long-lasting relief, thereby expanding the application scenarios of the atomizing liquid.

The above are merely some embodiments of the present application and do not limit the scope of the present application. Various modifications and variations can be made to the present application by those skilled in the art. Any modifications, equivalent substitutions, improvements, etc., made within the spirit and principles of the present application should be included within the scope of the present application.

What is claimed is:

1. An atomizing liquid modifier, comprising the following raw material components:
6-O-p-hydroxybenzoyl catalpol;
7α-hydroxy-β-sitosterol;
5,7-dihydroxy-6,8-dimethyl-3-(4′-hydroxy-3′,5′-dimethoxybenzyl)chroman-4-one;
3-hydroxy-citronellolic acid 3-O-β-D-glucopyranoside;
3′,4′,5,6,7,8-hexamethoxyflavone; and
an antioxidant.

2. The atomizing liquid modifier according to claim 1, wherein the antioxidant comprises resveratrol.

3. The atomizing liquid modifier according to claim 2, wherein, by mass parts, the atomizing liquid modifier comprises the following components:
0.10 to 1 parts of 6-O-p-hydroxybenzoyl catalpol;
0.1 to 1 parts of 7α-hydroxy-β-sitosterol;
0.1 to 0.5 parts of 5,7-dihydroxy-6,8-dimethyl-3-(4′-hydroxy-3′,5′-dimethoxybenzyl)chroman-4-one;
0.1 to 0.5 parts of 3-hydroxycitronellolic acid 3-O-β-D-glucopyranoside;
0.01 to 0.05 parts of resveratrol; and
0.01 to 0.05 parts of 3′,4′,5,6,7,8-hexamethoxyflavone and antioxidants.

4. A use of the atomizing liquid modifier according to claim 1 in the long-term relief of oral dryness caused by atomizing liquid.

5. An atomizing liquid, comprising the atomizing liquid modifier according to claim 1.

6. The atomizing liquid according to claim 5, further comprising glycerol and 1,2-propanediol.

7. The atomizing liquid according to claim 6, wherein a mass ratio of glycerol to the atomizing liquid modifier is 10:(0.084~0.62).

8. An aerosol generating device, comprising the atomizing liquid according to claim 5.
